(19) United States
(12) Patent Application Publication
(10) Pub. No.: US 2024/0358748 A1
(43) Pub. Date: Oct. 31, 2024

(54) AMORPHOUS DISPERSION OF FERRIC MALTOL AND THE PREPARATION PROCESS THEREOF

(71) Applicant: Indena S.P.A., Milano (IT)

(72) Inventors: Marilu' Tarantino, Milano (IT); Daniele Ciceri, Milano (IT); Maurizio Ricotti, Milano (IT)

(73) Assignee: Indena S.P.A., Milano (IT)

(21) Appl. No.: 18/578,076

(22) PCT Filed: Jul. 13, 2022
(86) PCT No.: PCT/IB2022/056445

§ 371 Date: Jan. 10, 2024

(30) Foreign Application Priority Data
Jul. 14, 2021 (IT) 102021000018578

(51) Int. Cl. A61K 33/26; A61K 9/16

(52) U.S. Cl. CPC A61K 33/26; A61K 9/1641; A61K 9/1694

(57) ABSTRACT

The invention relates to an amorphous solid dispersion comprising ferric maltol and a polymer, and the preparation process thereof.

### FIELD OF INVENTION

The invention relates to an amorphous solid dispersion comprising ferric maltol and a polymer, and the preparation process thereof.

### BACKGROUND TO THE INVENTION

Ferric maltol, having the formula 3-hydroxy-2-methyl-4H-pyran-4-one: iron (III) (3:1), the structure of which is shown in Formula 1, is a chemically stable complex of a ferric iron cation (Fe³⁺) and three maltol anions, which have the formula 3-hydroxy-2-methyl-4H-pyran-4-one.

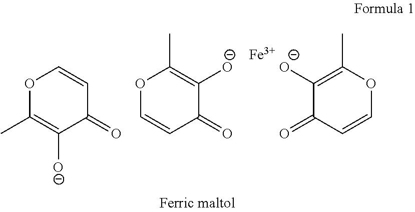

The chelating agent of the ferric cation (Fe³⁺) is maltol, an organic compound present in nature which is generally used in cookery as a flavour enhancer (Harvey R. S. et al., Aliment Pharmacol. Ther. 1998, 12 (9), 845-8); complexing gives rise to a ferric iron (Fe³⁺) in biologically assimilable form, which increases the absorption of elemental iron (Fe) by the muscle enterocytes.

The intake of ferric iron salts (Fe³⁺) usually involves little absorption of elemental iron (Fe) because, in the passage from the acid environment of the stomach to the more neutral environment of the duodenum, said salts are converted to insoluble hydroxypolymers (Pergola P.E. et al., Adv. Chronic Kidney Dis. 2019, 26 (4), 272-291) with a high affinity for intestinal mucus; said factors limit iron absorption. Conversely, in the case of ferric maltol, polymerisation is prevented by the presence of maltol, which acts as chelating agent (Barrand M.A. et al., J. Pharm. Pharmacol. 1987, 39 (3), 203-11). Moreover, the stabilising effect of maltol helps to minimise the amount of free iron in the intestine, thus helping to reduce side effects due to poor tolerability (Stallmach A. et al., Expert. Opin. Pharmacother. 2015, 16 (18), 2859-67).

Ferric maltol is currently used in the treatment of iron deficiencies, whether or not they are associated with anaemia; it is also effective and tolerated in the treatment of iron-deficiency anaemia in patients suffering from irritable bowel syndrome (Gasche C. et al., Inflamm. Bowel Dis. 2015, 21 (3),579-88); examples of products containing ferric maltol are Ferracru®, approved and marketed in Europe, and Accrufer®, approved and marketed in the USA.

WO 03/097627 and WO 2012/101442 relate to processes for the preparation of ferric maltol; the first patent application relates to a process for the preparation of ferric maltol from iron carboxylates in aqueous solution at a pH higher than 7, while the second patent application describes a process for the preparation of ferric maltol from non-carboxylated salts. Neither of the two applications mentions polymorphic forms of ferric maltol.

WO 2016/066555 discloses processes for the preparation of four different crystalline forms of ferric maltol. The process for the preparation of Forms I and II involves combining an aqueous solution of ferric citrate with an alkaline solution of maltol, using one of the crystals listed above as seed crystal. Form III is a solvate prepared by crushing Form I and/or Form II in a mixture of water and 1,4-dioxane. Form IV is obtained by crystallising Form I and/or Form II in one of the following solvents: 2-chlorobutane, methyl-t-butyl ether and 3-methylbutanol.

The solid form of a pharmacologically active ingredient is an important aspect to be taken into account when developing a medicament. Usually, a pharmacologically active ingredient can have a variety of solid forms, such as amorphous forms and polymorphic crystalline forms, having different physicochemical properties such as melting point, hygroscopicity, crystallinity, solubility and/or dissolution rate, bioavailability and storage stability, etc.; said properties must be taken into account when developing a medicament, because they may be relevant to the selection of the manufacturing or formulation process, transport and storage methods, etc.,

It is known that amorphous forms of pharmacologically active ingredients often exhibit better solubility and/or dissolution rates than the corresponding crystalline forms, with a consequent increase in bioavailability; said change in physicochemical properties is by no means insignificant but advantageous, because it allows to obtain other types of formulation than those used for crystalline forms, or to the development of therapeutic regimens wherein the amorphous active ingredient is used at lower doses.

Unfortunately, the amorphous form of ferric maltol obtained by the spray-drying technique or by rapid evaporation is unstable, and tends to revert to a more stable crystalline form. In the present invention, the term “spray-drying” refers to a process wherein a solution or suspension is pumped through a nozzle and ejected in the form of droplets into a drying chamber, wherein a stream of filtered hot air causes instant evaporation of the solvent in the droplets and at the same time directs the resulting solid towards a collector.

In view of the potential advantages of amorphous forms, there is still a pressing need to identify a stable amorphous form of ferric maltol and methods for obtaining it.

### DESCRIPTION OF THE FIGURES

FIG. 1: Diffractogram of the amorphous solid dispersion of ferric maltol according to Example 1.

FIG. 2: IR spectrum of the amorphous solid dispersion of ferric maltol according to Example 1.

FIG. 3: TG/DTA profile of the amorphous solid dispersion of ferric maltol according to Example 1.

FIG. 4: DSC profile of the amorphous solid dispersion of ferric maltol according to Example 1.

FIG. 5: Diffractogram of the amorphous solid dispersion of ferric maltol recorded after the stability study described in Example 2.

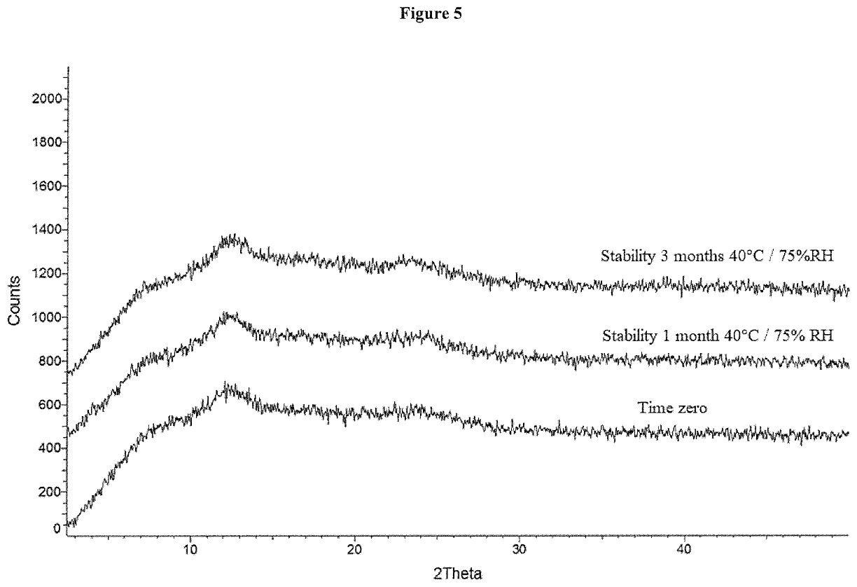

FIG. 6: Diffractogram of amorphous ferric maltol recorded after the stability study described in Example 2.

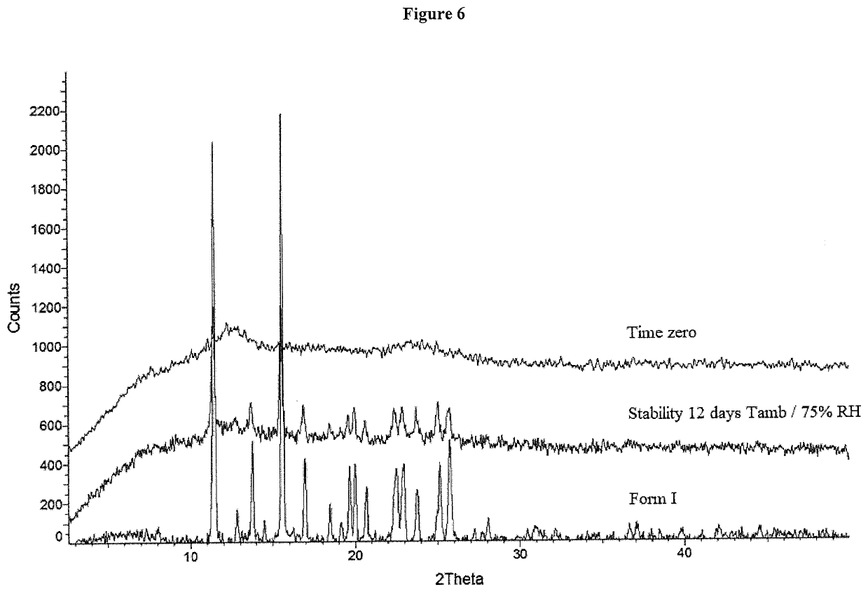

### DESCRIPTION OF THE INVENTION

The present invention relates to an amorphous solid dispersion comprising ferric maltol and a polymer, and the preparation process thereof.

In the present invention, the term “amorphous solid dispersion” is defined as a physically uniform mixture of two or more ingredients containing at least one ingredient in amorphous form and at least one further polymer; in particular, the ingredient in amorphous form is kinetically trapped (otherwise called “dispersed”) in the polymer in a non-crystalline high-energy state (Chavan R. B. et al., Asian J. Pharm, Sci. 2019, 14, 248-264).

In a first preferred aspect thereof, the invention relates to an amorphous solid dispersion of ferric maltol (ingredient in amorphous form) and a polymer, wherein the polymer is present in a smaller amount than the ferric maltol present in the dispersion; in particular, the polymer is present in an amount equal to or lower than 15%, 14%, 13%, 12%, 11%, 10%, 9%, 8%, 7%, 6%, 5%, 4%, 3%, 2% or 1% of the total weight of the dispersion. In the amorphous solid dispersions described in the prior art (Shamma R. N. et al., Powder Technology 2013, 237, 406-414; Ha Eun-Sol et. Al., Chem. Pharm. Bull. 2014, 62 (6), 545-551), the polymer is usually present in an amount equal to or greater than, preferably greater than, that of the ingredient in amorphous form. In fact, the presence of the polymer prevents crystallisation of the ingredient in amorphous form due to reduction of molecular mobility by increasing its glass transition temperature Tg, and/or due to the initiation of molecular interactions between the ingredients. In addition, the presence of the polymer increases the viscosity of the amorphous solid dispersion, thus reducing the nucleation rate, the coefficient of diffusion, and therefore the growth rate of the crystals. Said factors depend on the amount of polymer; for example, in the above-mentioned article by Ha Eun-Sol et al., it is demonstrated that as the amount of polymer increases, the solubility of the ingredient in amorphous form also increases. The fact that an amount of polymer equal to or lower than 15% of the total weight of the dispersion is sufficient in the amorphous solid dispersion according to the present invention is therefore an unexpected and surprising result. To clarify, in the amorphous solid dispersion according to the present invention, the amount of ferric maltol is equal to or greater than 85% and lower than 100%, and the amount of ferric maltol is preferably equal to or greater than 85%, 86%, 87%, 88%, 89%, 90%, 91%, 92%, 93%, 94%, 95%, 10 96%, 97%, 98% or 99% and less than 100%; again for the sake of clarity, in the amorphous solid dispersion according to the present invention, the sum of the amounts of ferric maltol and polymer is 100%.

A second aspect of the invention relates to a process (alternatively called “method”) for the preparation of an amorphous solid dispersion of ferric maltol wherein the polymer is present in an amount equal to or lower than 15% of the total weight of the dispersion; in particular, said process comprises at least one spray-drying step.

The amorphous solid dispersion of ferric maltol and a polymer according to the invention is characterised as follows:
I. the X-ray diffraction lattice (X-RPD) exhibits no recognisable peaks;
II. the Fourier-transform infrared spectrum (FT-IR) comprises the following absorption frequencies: 3066, 2917, 1733, 1563, 1500, 1458, 1272, 1198, 921, 848, 719 and 607 cm-1; in particular, the IR spectrum exhibits the following absorption frequencies: 3462, 3117, 3066, 2917, 2854, 1733, 1636, 1602, 1563, 1500, 1458, 1384, 1272, 1243, 1198, 1086, 1040, 974, 921, 848, 827, 763, 719, 607 and 560 cm-1;
III. the thermogravimetric and differential thermal analysis (TG/DTA) profile is characterised by a glass transition temperature ranging between 147° C. and 148° C., a recrystallisation peak with a maximum ranging between 183° C. and 184° C. followed by an unresolved endothermic peak with maxima at 273° C. and 280° C. respectively, due to melting with decomposition, and a weight loss amounting to 1.77% at the temperature of 120° C.;
IV. the differential scanning calorimetry (DSC) profile is characterised by a glass transition at about 140° C., a recrystallisation peak with a maximum at about 180° C., followed by an unresolved endothermic peak with a maximum at 281° C. due to melting with decomposition.

For the purposes of the present invention, “polymer” means one or more of the following: polyacrylic acids and polyacrylates, chitosan, cyclodextrins, maltodextrins, polymethacrylate, stearoyl macrogolglyceride, lactose, cellulose, methylcellulose, ethylcellulose, hydroxymethylcellulose, hydroxypropyl cellulose, hydroxypropyl cellulose acetate succinate, carboxymethylcellulose and the sodium salt thereof, cellulose acetate, cellulose acetate phthalate, cellulose hydroxypropyl methylphthalate, cellulose hydroxypropyl methylphthalate acetate succinate, polyvinyl alcohol, polyvinylpyrrolidone, polyvinylpyrrolidone vinyl acetate, polyethylene glycol, polyvinyl acetate phthalate, polyvinylpyrrolidone vinyl acetate copolymer, polyvinyl caprolactam-polyvinyl acetate-polyethylene glycol graft copolymer; the polymer is preferably a polyvinyl caprolactam-polyvinyl acetate-polyethylene glycol graft copolymer (hereinafter called “the graft copolymer”). Said graft copolymer is obtained by free-radical polymerisation of an N-vinyl-lactam, vinyl acetate and a polyether as described in U.S. Pat. No. 8,158,686, herein incorporated in full by reference. The graft copolymer is preferably obtained by polymerisation of polyvinyl caprolactam, polyvinyl acetate and polyethylene glycol; said copolymer is commercially available from BASF under the Soluplus® trademark.

Surprisingly, the solid dispersion according to the invention is stable for at least three months at a temperature of 40° C. under relative humidity conditions of 75%±5%: in fact, as demonstrated by the accelerated stability study described in Comparative Example 2, after 12 days at room temperature under relative humidity conditions of 75%±5%, ferric maltol, initially in completely amorphous form, tends to be converted, at least partly, to a crystalline structure characterised by the following distinctive reflections, expressed as 2θ° angles, amounting to 11.4, 13.7, 15.5, 16.9, 19.6, 19.9, 20.6, 22.4, 22.8, 23.7, 25.0, 25.7±0.2° 2θ. Said reflections are attributable to ferric maltol Form I (disclosed in WO 2016/066555). Conversely, the solid dispersion according to the present invention is in completely amorphous form not only after one month, but also after three months, at a temperature of 40° C. under relative humidity conditions of 75%±5%. The solid dispersion according to the invention is typically prepared by a process comprising the following steps:
a) ferric maltol and polymer are suspended in an organic solvent to give a suspension (suspension A);
b) suspension A is maintained under reflux and under stirring until a solution (solution B) is obtained;
c) solution B is cooled to room temperature and filtered to give a further solution (solution C);
d) the solvent of solution C is removed by spray-drying.

Typically, the weight ratio used between ferric maltol and the polymer in step a) is in the 5-99 range; preferably, in the 9-19 range.

Typically, the organic solvent used in step a) is a solvent selected from the following: methanol, ethanol, propanol, isopropanol, butanol, acetic acid, formic acid, dichloromethane, ethyl acetate, acetone, dimethylsulphoxide, dimethylformamide, tetrahydrofuran or mixtures thereof. According to a preferred embodiment, the solvent is methanol.

### Experimental Section

### Materials and Methods

Soluplus®, commercially available from BASF, was used as graft copolymer.

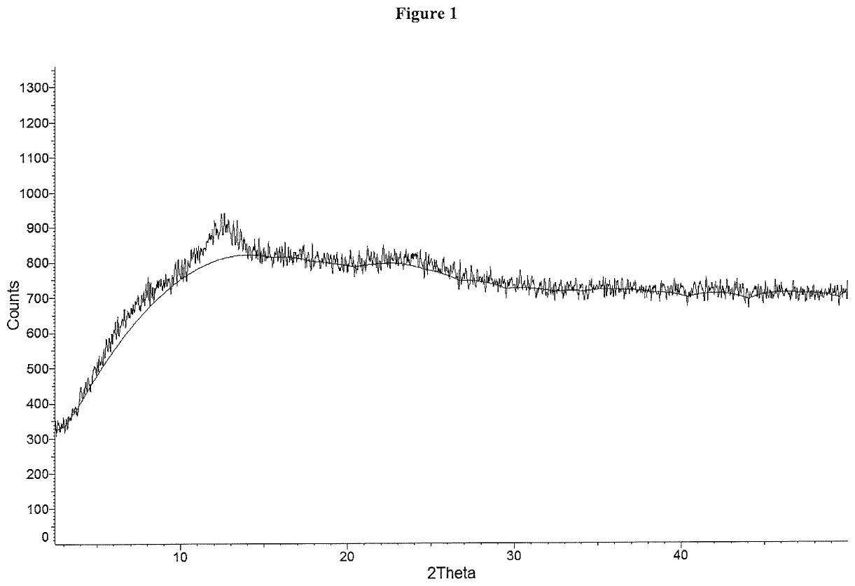

### X-Ray Powder Diffraction (FIG. 1)

The X-ray diffraction pattern was recorded on a Bruker D2-Phaser diffractometer. The X-ray generator was set to 30 kV and 10 mA, using Cuk as radiation source. The sample was prepared in a sample holder and irradiated for an irradiation length of 10 mm. The data were recorded between 2 and 50 2θ degrees every 0.02 2θ degrees, with a recording time of 3 seconds per 2θ degree.

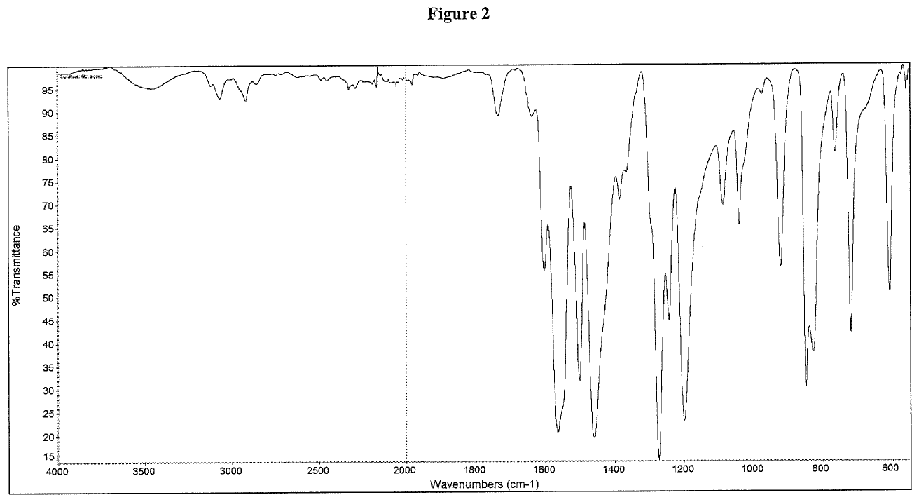

### Fourier-Transform Infrared Spectroscopy (FTIR) (FIG. 2)

The infrared spectrum was recorded in attenuated total reflectance (ATR) using the Nicolet iS10 (Thermo Fisher) Fourier-transform spectrometer, equipped with the Specac ATR Golden Gate accessory. The spectrum results from the acquisition and transformation of 32 scans in the spectral region between 4000-500 cm⁻¹ at a resolution of 4 cm⁻¹.

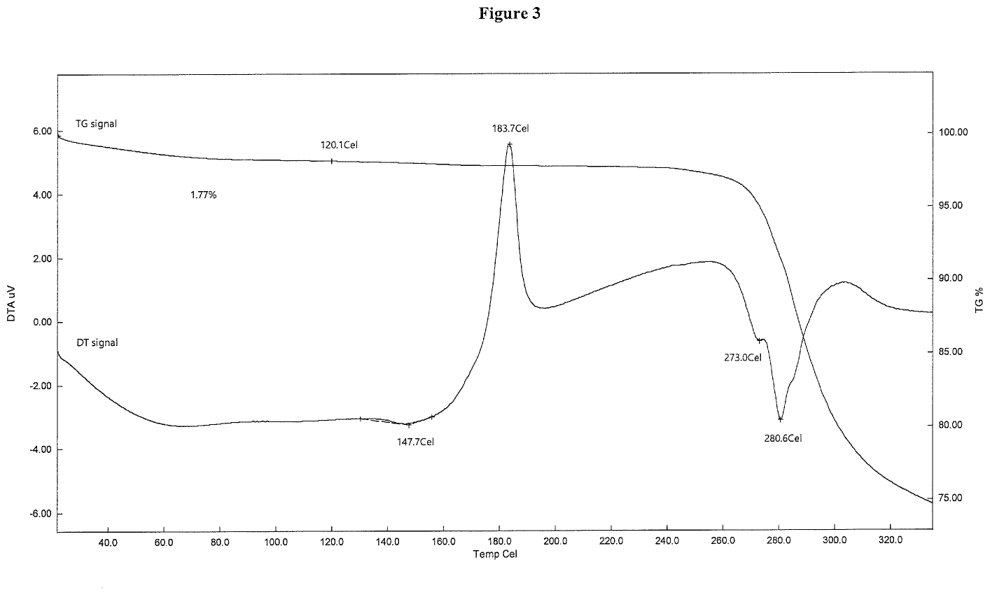

### Thermogravimetry (TG) and Differential Thermal Analysis (DTA) (FIG. 3)

The analysis was conducted with a Hitachi TG/DTA7200 instrument in open aluminium crucibles (volume 40 μL). The TG/DT signal was recorded between 30° C. and 300° C. with a linear heating gradient (10° C./min) under nitrogen flow (200 mL/min). About 10 mg of sample was used for the measurement.

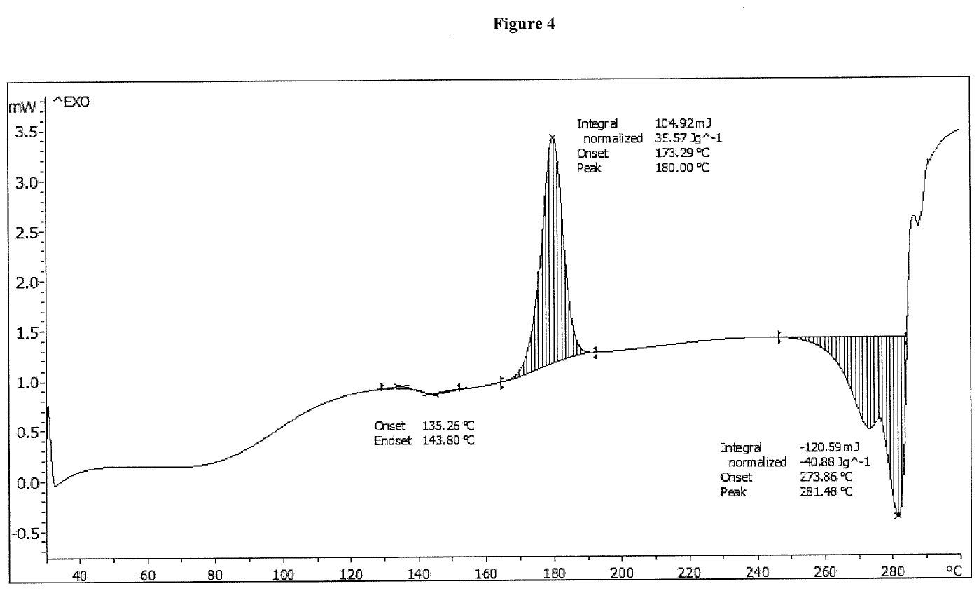

### Differential Scanning Calorimetry (DSC) (FIG. 4)

The analysis was conducted with a Mettler DSC1 System instrument. The heat flow was recorded in a range between 30° and 300° C. with linear gradient (10° C./min) and under nitrogen flow (50 mL/min). About 5 mg of sample was used for the measurement, in a sealed and then perforated aluminium crucible (volume 40 μl).

### EXAMPLES

### Example 1—Preparation of an Amorphous Solid Dispersion of Ferric Maltol

9 g of ferric maltol and 1 g of Soluplus® were suspended in methanol (450 mL). The suspension was maintained under stirring and under reflux until the ferric maltol and Soluplus® were completely dissolved, for a time of 30 minutes. The resulting solution was brought to room temperature, filtered and dried using a spray-dryer.

The spray-drying was conducted with a Buchi laboratory Mini Spray Dryer B-290™ connected to the Buchi Inert Loop B-295™ accessory. The operating conditions used are as shown in Table 1:

TABLE 1

| | | |
|---|---|---|
| Input temperature | 140° | C. |
| Output temperature | 80° | C. |
| Spray-drying pressure | 0.41 | bar |
| Qflow | 40 | mm |

| | |
|---|---|
| Feed rate | 13% |
| Suction | 96% |

The resulting solid was further dried for 18 hours at 50° C. under vacuum. The amorphous solid dispersion is obtained as a soft, reddish-orange powder.

### Example 2—Accelerated Stability Study (Comparison Between the Stability of the Amorphous Solid Dispersion of Example 1 and Ferric Maltol in Amorphous Form)

The amorphous solid dispersion prepared according to Example 1, and ferric maltol in amorphous form, prepared according to the procedure reported in Example 1 but without the addition of Soluplus®, underwent an accelerated stability study. It was observed in the study that after only 12 days at room temperature and relative humidity of 75%±5%, ferric maltol exhibits the following reflections, expressed as 2θ° angles: 11.4, 13.7, 15.5, 16.9, 19.6, 19.9, 20.6, 22.4, 22.8, 23.7, 25.0, 25.7±0.2° 2θ, attributable to ferric maltol Form I. Conversely, the amorphous solid dispersion according to the present invention, after one month and three months at a temperature of 40° C. and relative humidity of 75%±5%, is still in amorphous form, not exhibiting any recognisable peak.

1. An amorphous solid dispersion comprising ferric maltol and a polymer.

2. The solid dispersion according to claim 1, wherein the polymer is selected from the following: polyacrylic acids and polyacrylates, chitosan, cyclodextrins, maltodextrins, polymethylacrylate, stearoyl macrogolglyceride, lactose, cellulose, methylcellulose, ethylcellulose, hydroxymethylcellulose, hydroxypropylcellulose, hydroxypropylcellulose acetate succinate, carboxymethylcellulose and the sodium salt thereof, cellulose acetate, cellulose acetate phthalate, cellulose hydroxypropylmethylphthalate, cellulose hydroxypropylmethyl acetate succinate, polyvinyl alcohol, polyvinylpyrrolidone, polyvinylpyrrolidone vinyl acetate, polyethylene glycol, polyvinyl acetate phthalate, polyvinylpyrrolidone vinyl acetate copolymer, polyvinyl caprolactam-polyvinyl acetate-polyethylene glycol graft copolymer.

3. The solid dispersion according to claim 2, wherein the polymer is a polyvinyl caprolactam-polyvinyl acetate-polyethylene glycol graft copolymer.

4. The solid dispersion according to claim 1, wherein the polymer is present in a smaller amount than the ferric maltol present in the dispersion.

5. The solid dispersion according to claim 1, wherein the polymer is present in an amount equal to or less than 15% of the total weight of the dispersion.

6. The solid dispersion according to claim 4, wherein the polymer is present in an amount equal to or less than 10% of the total weight of the dispersion.

7. The amorphous solid dispersion according to claim 1, characterized by an IR spectrum comprising the following absorption frequencies: 3066, 2917, 1733, 1563, 1500, 1458, 1272, 1198, 921, 848, 719 and 607 cm-1.

8. The amorphous solid dispersion according to claim 7, characterised by an IR spectrum comprising the following absorption frequencies: 3462, 3117, 3066, 2917, 2854, 1733, 1636, 1602, 1563, 1500, 1458, 1384, 1272, 1243, 1198, 1086, 1040, 974, 921, 848, 827, 763, 719, 607 and 560 cm-1.

9. The solid dispersion according to claim 1, wherein the thermogravimetric and differential thermal analysis (TG/DTA) profile is characterised by a glass transition ranging between 147° C. and 148° C., a recrystallisation peak with a maximum ranging between 183° C. and 184° C. followed by an unresolved endothermic peak with maxima at 273° C. and 280° C. respectively due to melting with decomposition, and a weight loss of 1.77% at the temperature of 120° C.

10. The solid dispersion according to claim 1, wherein the differential scanning calorimetry (DSC) profile is characterised by a glass transition at about 140° C., a recrystallisation peak with a maximum at about 180° C. followed by an unresolved endothermic peak with a maximum at 281° C. due to melting with decomposition.

11. A process for preparing a solid dispersion according to claim 1 comprising at least one spray-drying step.

12. The process according to claim 11 comprising the following steps:
a) suspending amorphous ferric maltol and polymer in an organic solvent, to give a suspension A;
b) keeping suspension A under reflux and under stirring until a solution B is obtained;
c) cooling solution B to room temperature and filtering said solution B to obtain solution C;
d) removing the solvent of solution C by spray-drying.

13. The process according to claim 12, wherein the weight ratio of ferric maltol to polymer ranges between 5 and 99.

14. The process according to claim 12, wherein the weight ratio of ferric maltol to polymer ranges between 9 and 19.
